(19) United States
(12) Patent Application Publication
(10) Pub. No.: US 2003/0028255 A1
(43) Pub. Date: Feb. 6, 2003

(54) Bioabsorbable exoluminal stent

(76) Inventors: Gregory K. Hartig, Cross Plains, WI (US); Nadine P. Connor, Madison, WI (US); Sarvi Singh Nalwa, Milwaukee, WI (US); Gregory K. Sewall, Madison, WI (US)

Correspondence Address: DEWITT ROSS & STEVENS S.C., 8000 EXCELSIOR DR, SUITE 401, MADISON, WI, 53717-1914, US

(21) Appl. No.: 10/102,494

(22) Filed: Mar. 19, 2002

Related U.S. Application Data
(60) Provisional application No. 60/290,431, filed on May 12, 2001.

(51) Int. Cl. A61F 2/04

(52) U.S. Cl. 623/23.7; 623/23.71

(57) ABSTRACT

Disclosed is an exoluminal stent. The stent includes a bioabsorbable matrix having a plurality of apertures passing therethrough. The matrix is capable of being dimensioned and configured to complement an external diameter of a vessel to be stented. The stent can be adhered to the outer diameter of the vessel to be stented via sutures that pass through the apertures in the matrix.

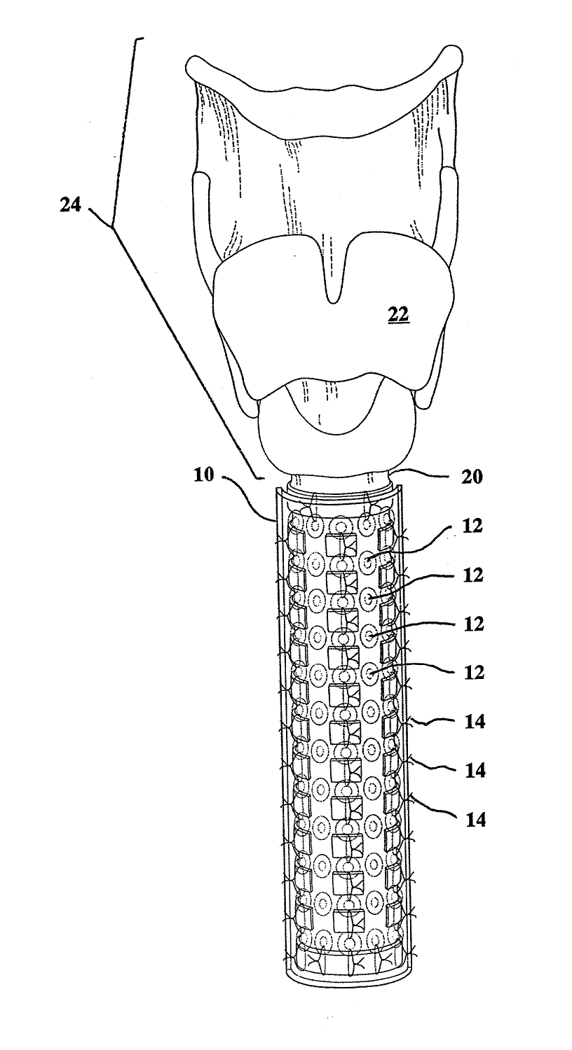

Priority is hereby claimed to provisional application Serial No. 60/290,431, filed May 12, 2001, the content of which is incorporated herein by reference.

### FIELD OF THE INVENTION

The invention is directed to bioabsorbable, exoluminal stents. The stents described herein can be used for the repair of tracheomalacia.

### BACKGROUND

Tracheomalacia is a congenital or acquired condition characterized by tracheal cartilage flaccidity that results in airway collapse during expiration. Symptoms range from mild expiratory stridor to apneic episodes with cyanosis and bradycardia. In mild pediatric cases, this problem may resolve without intervention as the trachea grows larger, firmer, and less prone to collapse over time. However, in more severe cases, surgical intervention is required, as the associated airway collapse can lead to progressive respiratory compromise and a potentially death.

Traditional methods of tracheomalacia repair include aortopexy and tracheal resection. Although effective under certain conditions, these techniques possess significant limitations. The use of aortopexy is limited to situations in which the malacic segment is opposite the aorta. Tracheal resection is only applicable to very short malacic segments and is often complicated by stenosis at the anastomosis site.

Within the last decade, external and internal airway stenting techniques have been attempted for tracheomalacia repair, but have not proven to be consistently reliable. Limitations of current methods of stenting include the lack of a shape-memory material that is easily inserted, accommodates airway growth in children, is non-reactive to tissues, and is easily removed. Due to the small size of the pediatric airway, internal stenting has been associated with severe complications, including stent migration and death. The lack of a suitable graft material, synthetic or autologous, has limited the efficacy of external stenting See, for example, Mair et al. (1991) Laryngoscope, 101:1002-1008; Loef et al, (1988) Journal of Pediatric Surgery, 23:1173-1177; Tsugawa et al. (1997) Journal of Pediatric Surgery 32:50-53; and Shaha et al (1991) American Journal of Surgery, 162:417-420.

Tracheomalacia can be associated with a variety of congenital anomalies, including cardiovascular defects, developmental delay, esophageal anomalies, and gastroesophageal reflux. Tracheomalacia can be caused by a diffuse process of congenital origin or by a localized abnormality such as a vascular ring, anomalous innominate artery, esophageal atresia, or tracheoesophageal fistula. Internal compression by an endobronchial or tracheostomy tube may also result in tracheomalacia. Tracheomalacia, however, is rarely found in combination with laryngomalacia.

Tracheomalacia most commonly affects the distal one-third of the trachea. By virtue of its normal flexibility or compliance, the trachea changes caliber during the respiratory cycle. Tracheal dilatation and lengthening occurs during inspiration; narrowing and shortening occurs during expiration. Accentuation of this cyclic process may cause excessive narrowing of tracheal lumen, thus deforming the entire length or a localized segment of the trachea.

The functional impairment caused by tracheomalacia is proportional to the length of the involved segment and the degree of stenosis. Furthermore, kinking may occur at the transition between normal tracheal wall and indurated segments as well as in the malacic segment. In diffuse tracheal disease or when extensive peritracheal adhesions are present, the trachea usually distends unevenly during inspiration and collapses during expiration, thus interfering with the tracheal function.

Anatomically, the human trachea commences at the cricoid cartilage and terminates at fifth thoracic vertebra. It lengthens and dilates during inspiration and narrows and shortens during expiration. Fifteen to 20 incomplete rings of cartilage prevent it from collapsing. The trachea is separated from the vertebral column by the esophagus posteriorly. In the thorax, the jugular venous arch lies anteriorly to the trachea at the sternum; the brachiocephalic trunk and left common carotid artery lie at the level of the third thoracic vertebra. The arch of aorta is to the left and front of the distal trachea just before it bifurcates. On the right of the trachea are pleura, on the left is the aortic arch, and posterolaterally is the left subclavian artery.

The relation of the trachea to the aortic arch makes it liable to compression from aneurysm or from vascular rings, which occur with abnormal arterial development. Therefore, for distal tracheomalacia, whether associated with tracheoesophageal fistula or with vascular anomalies, aortopexy is the procedure of choice for surgical correction of tracheomalacia. This procedure anchors the aorta to the sternum, thus preventing the aorta from exerting pressure on the trachea.

A definitive diagnosis of tracheomalacia depends on an accurate medical history combined with proper endoscopic evaluation. The airway is directly visualized during spontaneous respiration using ventilating laryngoscopy and telescoping bronchoscopy. Flexible bronchoscopy may also be utilized. Findings consistent with a diagnosis of tracheomalacia include the following classic triad: loss of the normal semicircular-shape of tracheal lumen; forward ballooning of the posterior membranous wall; and anteroposterior narrowing of the tracheal lumen.

A number of internal, non-absorbable tracheal stents are available commercially. For example, Flexstent Medical Corporation, of Jiangsu, China, markets self-expanding internal tracheal stents. Medilyfe of Montreal, Canada, is also a commercial supplier of internal tracheal stents These types of internal stents, however, present certain inherent and insurmountable risks and drawbacks. For example, migration of the stent can exacerbate the previously-existing malacia. Migration of the stent might also require emergency removal of the stent. Infection at the point of deployment and/or in-growth around the internal stent can result in undesirable narrowing of the tracheal lumen. Also, the stent must ultimately be removed or the trachea will be permanently narrowed at the point of stent deployment.

Several forms of internal and external stenting for tracheomalacia have recently been evaluated in animal models, including woven polymeric stents, stainless steel “zig-zag” stents, and thermal shape-memory titanium-nickel alloy internal stents. See Mair et al. (1991) Laryngoscope 101:1002-08; Radlinsky et al. (1997) Vet. Surg. 26:99-107; and Furman et al. (1999) Arch. Otolaryngol. Head Neck Surg. 125:203-7.

However, these methods are associated with less than optimal outcomes, such as severe respiratory obstruction with inflammation and tracheal stenosis, and death due to stent migration. Further, histopathologic examination revealed squamous metaplasia of the stented lumen. Metallic angioplasty stents have also been implanted in the trachea and bronchi of pediatric patients, with good results in only 4 of 7 children over a mean follow-up duration of 11 months. See Robey et al. (2000) Laryngoscope 1(10):1936-42.

External stenting has included the use of non-absorbable polytetrafluoroethylene (PTFE) grafts (Filler et al. (1998) J. Ped. Surg. 33:304) and non-absorbable polypropylene mesh secured by suture or fibrin glue (Vinograd et al. (1987) J. Surg. Res. 42:597-604). These stents have also been associated with complications, including death due to graft erosion and fixation of the trachea around its entire circumference. As a result, these techniques have not gained widespread acceptance for the surgical management of pediatric tracheomalacia.

### SUMMARY OF THE INVENTION

The invention is directed to an exoluminal stent made of a bioabsorbable material. The stent allows for external stenting of a vessel, such as the trachea, early in life without creating a long-term impediment to growth of the stented vessel. Further, an absorbable stent obviates the need for surgical removal of the stent, thus eliminating the presence of a longstanding foreign body capable of infection or erosion. This is especially so in the pediatric population, where a temporary stent appears ideal. For example, in the case of pediatric tracheomalacia, the overwhelming majority of patients develop increased tracheal stability with age, and thus no longer require tracheal stenting.

Thus, the invention is directed to an exoluminal stent comprising a bioabsorbable matrix having a plurality of apertures passing therethrough. The matrix is capable of being dimensioned and configured to complement an external diameter of a vessel to be stented. Thus, in the preferred embodiment, the vessel to be stented is exposed and measured, the stenting material is shaped, usually at elevated heat, to complement the outer diameter of the vessel. The stent is then adhered to the outer diameter of the vessel, using, for example, sutures passing through the apertures or using a glue (such as fibrin glue). The surgical would is then closed. Over time, the exoluminal stent is absorbed by the body.

In the preferred embodiment of the stent, the absorbable matrix comprises a poly(lactic acid)/poly(glycolic acid) co-polymer.

The invention is also drawn to a corresponding method externally stenting a vessel in need thereof. The method comprises adhering a bioabsorbable matrix having a plurality of apertures passing therethrough to a vessel to be stented, wherein the matrix is dimensioned and configured to complement the external diameter of the vessel. In the preferred embodiment of the method, the vessel to be stented is a human trachea.

### BRIEF DESCRIPTION OF THE DRAWINGS

The sole drawing FIGURE is a rendering of an exoluminal stent 10 according to the present invention. The stent is deployed about a human trachea 20.

### DETAILED DESCRIPTION OF THE INVENTION

In the majority of cases, tracheomalacia in the pediatric population is mild and is often managed conservatively. In severe cases, however, surgical intervention is required. Because of the limitations of aortopexy and tracheal resection, airway stenting has gained increased attention for the management of severe tracheomalacia. However, internal stenting has been associated with significant limitations that have precluded routine clinical application for this condition.

The ideal pediatric airway stent would possess the following features: (a) adequate strength to prevent airway collapse, (b) technical ease of placement, (c) accommodation of future airway growth, (d) lack of significant tissue reactivity, and (e) bioabsorbability, thereby precluding the need for a second procedure to remove the stent. In the present invention, it has been found that poly(lactic acid)/poly(glycolic acid) co-polymer absorbable plates exhibited all of these features.

Thus, the present invention addresses these concerns by providing an exoluminal, absorbable stent. The stent is sufficiently stiff to prevent airway collapse; it is easily deployed using standard surgical techniques; because the stent degrades over time, it allows for future growth of the stented vessel; and the stent material does not exhibit adverse biological interactions.

In the Example presented herein, the use of PLPG absorbable plates as external stents resulted in the immediate repair of a potentially lethal tracheomalacia in all six animal models subjected to the treatment. Stent placement was technically simple, and the stents were found to be stable, without evidence of migration or tracheal erosion. Tissue reaction within the trachea was minimal, and ciliated respiratory epithelium was largely maintained. These plates, typically used for craniomaxillofacial fixation, undergo progressive resorption with 90% loss of strength within 8 weeks, and complete resorption within one year.

In the animal models presented in the Example, follow-up examinations performed 9-12 weeks after stent deployment revealed that none of the 6 animals in the Example showed signs or symptoms of recurrent malacia.

This invention also presents the first attempt to quantify the effects of tracheal stenting on airway growth. In the animal model, inner tracheal diameter within the stented segment (tracheal ring 4) was 2-4 mm less than the diameter within normal rings above and below this segment (rings 1 and 8). This decrease in inner tracheal diameter ranged from 14 to 33%, and was clinically insignificant, as all animals displayed the absence of respiratory distress and showed rapid weight gain, findings clearly indicating the good health and adequate respiratory status of the animals. This narrowing may be partially attributed to the rapid growth rate in our animal model. During the 9 to 12 week study period, these animals displayed an average weight gain of 29 kg and an average increase in outer tracheal diameter of 6.5 mm at ring 8. In a pediatric patient, the PLPG stent would be completely resorbed before such growth was achieved. Accordingly, less restriction of airway growth would be expected.

Congenital tracheomalacia is often characterized by widening of the membranous posterior wall in addition to cartilaginous instability. The porcine model employed in the Example could not recreate membranous wall widening. However, the model clearly results in tracheal collapse, with the same approximation of the anterior and posterior tracheal walls as is seen in congenital malacia.

As used herein, the term “absorbable” or “bioabsorbable” refers to any suitably stiff material (i.e., a material sufficiently stiff to stent the vessel to which it is attached) that will be absorbed when implanted within an animal body. The adjective “absorbable” also explicitly indicates that the material is non-toxic to the animal host, degrades into non-toxic products, and will not precipitate a life-threatening immunogenic reaction upon implantation. The material may provoke less serious, non-life threatening adverse reactions (immunogenic or otherwise, such as minor inflammation, irritation, and the like) and still be considered “absorbable” as that term is defined herein. An absorbable material, as defined herein, will maintain its structural integrity and rigidity for up to 24 months after implantation. At a minimum, an absorbable material, as defined herein, will retain its structural integrity and rigidity for at least 30 days after implantation. In the preferred embodiment, the absorbable material is of a suitable composition and thickness that it retains its structural integrity and rigidity for approximately six months after implantation, and then gradually degrades and is fully absorbed by the host within 18 months after implantation.

The term “heat-labile” as used herein refers to materials that are stiff or slightly flexible, and retain their shape at temperatures slightly above, at, and below the normal body temperature of the animal host, but that can be softened and molded into a desired shape at temperatures significantly higher than the normal body temperature of the host. Thus, as used herein, a heat-labile, absorbable material is a material that can be molded into a desired shape at elevated temperature, stiffens when cooled to the normal body temperature of the host animal, and will remain still when initially implanted into the host.

It is much preferred that the absorbable material used in the present invention be heat-labile. This is not a requirement of the present invention, but is preferred.

More specifically, the preferred materia for use in the invention is poly-L-lactic acid/polyglycolic acid co-polymers (referred to hereinafter as PLPG). (As used herein, the term “copolymer” designates all copolymers of any description, including block copolymers and graft copolymers.) This material can be cast into heat-labile, moldable plates of virtually any dimension or thickness. This material has many highly desirable characteristics, including its strength, versatile shaping characteristics, and resorbability. Thus, for example, PLPG plates allow for temporary, exoluminal stenting of the trachea (or other lumen) while simultaneously allowing for airway growth and eliminating the need surgical removal.

Suitable PLPG plates can be obtained commercially from Biomet, Inc. Warsaw, Ind., under the trademark LactoSorb®. LactoSorb®-brand copolymer is an absorbable co-polymer synthesized from 82% L-lactic acid and 18% glycolic acid. LactoSorb®-brand copolymer is substantially amorphous (i.e., without crystallinity), thus its degradation and absorption within the animal body tends to be uniform throughout the mass of the implanted stent.

When implanted with an animal host, PLPG co-polymers having this approximate ratio (82% lactic acid/18% glycolic acid) will retain most of its strength for six to eight weeks of substantially longer if the stent is fabricated from a thicker plate of material. Mass loss, which generally follows strength loss for absorbable polymers, occurs in roughly twelve months (approximately) for the LactoSorb@-brand copolymer.

Adjusting the composition of the copolymer enables bioabsorbability to be regulated. Thus, a 50%-50% PLPG co-polymer tends to degrade and be absorbed more quickly than an 80%-20% PLPG co-polymer.

Other biodegradable polymers can also be used in the invention with equal success. For example, poly(lactic acid) (PLA) (synonymous with polylactide) and poly(glycolic acid) (PGA) (synonymous with polyglycolide) homopolymers are also suitable for use in the present invention. PLA and PGA homopolymers are both available commercially from Purac Biochem (Netherlands). Note also that any of the various stereo forms of PLA (poly-L-lactic acid, poly-D-lactic acid, stereo copolymers of poly-L and poly-D-lactic acid) will function with equal success in the invention. This is also true of the PLPG co-polymers: the lactic acid monomers of the preferred PLPG co-polymer can be in the L or D configuration or a mixture of L and D monomers.

A host of PLPG copolymers of various ratios are available commercially. Purac Biochem and Biomet, noted above, supply PLPG copolymers of various compositions. Additionally, Ethicon, of Sommerville, N.J. formulates VICRYL®-brand suture material from 10%-90% PLA to PGA. Such material, when provided in the form of plates, is suitable for use in the present invention.

Another bioabsorbable material that can be used in the present invention are poly(amino ester) polymers, such as desaminotyrosyltyrosine ethyl ester (i.e., poly (DTE carbonate)), also referred to as tyrosine polycarbonates. For an example of this class of bioabsorbable polymer, see U.S. Pat. No. 6,120,491. In short, these polymers have the general structure:

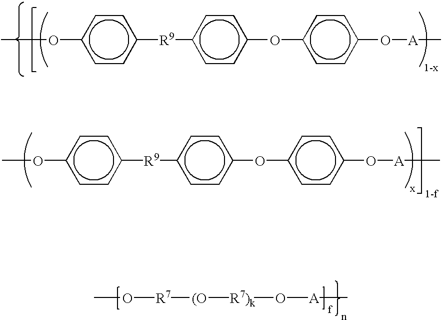

where R⁹ is an alkyl, aryl or alkylaryl group with up to 18 carbon atoms having a pendent carboxylic acid group or the benzyl ester thereof; R¹² is an alkyl, aryl or alkylaryl group with up to 18 carbon atoms having a pendent carboxylic acid ester group selected from straight and branched alkyl and alkylaryl esters containing up to 18 carbon atoms and ester derivatives of biologically and pharmaceutically active compounds covalently bonded thereto, provided that the ester group is not a benzyl group or a group that is removed by hydrogenolysis; each R⁷ is independently an alkylene group containing up to four carbon atoms; A is selected from:

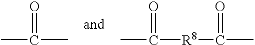

wherein R⁸ is selected from saturated and unsaturated, substituted and unsubstituted alkyl, aryl and alkylaryl groups containing up to 18 carbon atoms; k is between about 5 and about 3,000; and x and f independently range from zero to less than one.

Other suitable bioabsorbable materials that can be used in the invention include poly(caprolactones) and polydioxanones (e.g., poly(p-dioxanone). Sheets of these materials can be obtained commercially from a number of suppliers, such as Ethicon.

Copolymers these materials are also suitable for use in the present invention. Thus, for example, copolymers of ε-caprolactone and L-lactide are elastomeric when prepared from about 25% ε-caprolactone and about 75% L-lactide and quite rigid when prepared from about 10% ε-caprolactone and about 90% L-lactide. Similarly, copolymers of ε-caprolactone and glycolide (i.e., poliglecaprone) are sufficiently rigid to be used in the present invention.

The thickness of the absorbable plate generally should range anywhere from about 0.25 mm to 10 mm. The thicker the plate, the longer it will remain in the body after implantation. The medical practitioner may select the width deemed most suitable for the lumen being stented based upon the age and general physical condition of the patient, the accessibility of the area to be stented, etc. For example, where the trachea is being stented, it is generally believed that a plate of approximately 1 mm in thickness is appropriate. The invention does, however, encompasses stents having a thickness greater than or less than the 0.25 to 10 mm thickness note hereinabove.

The stent also includes a plurality of apertures passing therethrough. These apertures can be used to adhere the stent to the vessel to be stented using conventional absorbable sutures. The stent may also be adhered to the vessel using a suitable surgical glue, such as fibrin glue. Sutures, however, are preferred because of their sure and steadfast placement of the stent.

Referring now to the sole drawing FIGURE, the FIGURE is an anterior view of an excised human larynx 24 and trachea 20, with stent 10 deployed about the trachea via sutures 14 passing through apertures 12. The thyroid cartilage is shown at 22. The stent 10 thus functions to prevent the underlying trachea 20 from collapsing.

In practice, deploying the subject stent is well within the surgical skills of a licensed surgeon. When stenting a trachea, for example, the malacic segment is exposed, the length and the diameter of the segment to be stented is determined, and a plate of absorbable material is cut to the appropriate size. The plate is then heated to render it malleable. Once malleable, the plate is molded to be complementary in shape to the outer diameter of the lumen to be stented. Thus, as a general rule, the plate will be fabricated into a roughly cylindrical shape, a U-shaped segment, or a C-shaped segment, etc. A mandrel can be used for this purpose. For sake of speed during surgery, the plates may also come pre-shaped in assorted sizes, lengths, diameters, etc., to fit pediatric patients, geriatric patients, etc.

The stent is then deployed about at least a portion of the outer diameter of the vessel to be supported and sutures (or staples or any other type of suitable fastener) are passed through the apertures in the stent and through at least the outermost layer of the vessel being stented. In this fashion, the vessel being stented is gently urged against the inside diameter of the exoluminal stent, thereby opening the lumen of the vessel.

### EXAMPLE

The following Example is included solely to aid in a more complete understanding of the invention described and claimed herein. The Example does not limit the scope of the invention in any fashion.

Six pigs weighing from 16 to 25 kg were used. The pig was selected as the animal model due its established use in previous studies of tracheomalacia and its rapid airway growth. See, for example, Mair et al. (1991) Laryngoscope, 101:1002-1008; and Johnston et al. (1980) The Annals of Thoracic Surgery, 30:291-296. Rapid airway growth in this animal model allowed for examination of the efficacy of surgical repair after substantial increases in tracheal diameter had occurred. All animals were treated in accordance with guidelines established by the University of Wisconsin Research Animal Resources Center (RARC).

The animals were sedated intramuscular injection of telazol, xylazine, and atropine. Each pig was then endotracheally intubated, and general anesthesia was achieved with inhalational isoflurane. The trachea was exposed via a midline neck incision. The anterior and lateral aspects of the trachea were separated from the strap muscles, thyroid gland, and overlying soft tissues by blunt dissection. The external tracheal diameter was measured at rings 1, 4, and 8.

This animal model was based on a previous study by Mair et al., supra, in which resection of five consecutive tracheal rings resulted in a fatal malacia. In this Example, a malacic tracheal segment was created by resecting the submucosa of three interrupted segments of cartilage from six consecutive tracheal rings (rings 2-7). Entire tracheal rings were not removed in order to create a model that more closely resembled pediatric tracheomalacia, in which tracheal cartilage is present, but lacks adequate strength to prevent airway collapse.

Each pig was then examined during spontaneous ventilation, and endoscopic photographs and videotapes were taken to document the extent of airway collapse in the animals. Photographs were taken using a flexible endoscope inserted through the endotracheal tube after withdrawing the tube to a point proximal to the malacic segment. The severity of tracheomalacia was documented by noting the presence of stridor, retractions, and cyanosis during spontaneous ventilation. Mechanical ventilation and general anesthesia were then resumed.

A PLPG mesh panel (50×50 mm, 0.75 mm thickness) was cut to a size slightly larger than the malacic tracheal segment. The plate was fashioned into a rough “U” shape to recreate the normal tracheal contour. Using 4-0 and 5-0 absorbable polyglycolic acid sutures, the stent was then secured to the underlying airway. The airway was reexamined during spontaneous ventilation, and endoscopic photographs were taken. Each animal was then reevaluated for the presence of stridor, retractions, or cyanosis during spontaneous ventilation.

Animals were followed for 9 to 12 weeks postoperatively. Daily evaluations for signs of respiratory distress and oral intake were performed, and weight gain was recorded. At the completion of the observation period, repeat bronchoscopy with photography of the repair site was performed during spontaneous ventilation. The animals were then euthanized, and the larynx and cervical trachea were harvested. The inner and outer tracheal diameters at rings 1, 4, and 8 were measured.

Each trachea was then fixed in 10% neutral buffered formaldehyde and sectioned in the sagittal and transverse planes. Decalcification in acidic solutions did not soften the prosthetic material, the trabeculae of which were removed from fixed tissue sections with a fine forceps. Tissue sections, 5 microns in thickness, were then stained with hematoxylin and eosin and examined using light microscopy.

Following creation of the malacic segment, all six pigs developed stridor, substernal and intercostal retractions, and cyanosis during spontaneous ventilation. Endoscopic examination revealed near-total collapse of the involved tracheal segment in all six animals. After repair, repeat endoscopy revealed the absence of tracheal collapse during spontaneous ventilation. All animals were successfully extubated without evidence of postoperative respiratory distress. The follow-up period ranged from 9 to 12 weeks. During this period, there was no evidence of respiratory distress upon daily examination of all six animals, and all animals showed rapid weight gain (see Table 1). Follow-up endoscopic examination revealed the absence of tracheal collapse during spontaneous ventilation and no evidence of reactive changes within the tracheal lumen.

TABLE 1
DURATION OF POSTOPERATIVE
OBSERVATION PERIOD AND ANIMAL WEIGHT GAIN

| Pig | Observation Period | Weight Gain (kg) |
|---|---|---|
| 1 | 9 wk 3 d | 25.4 |
| 2 | 10 wk 5 d | 26.8 |
| 3 | 10 wk | 26.1 |
| 4 | 12 wk | 43.2 |
| 5 | 10 wk | 26.8 |
| 6 | 9 wk | 25.5 |

At post-mortem examination, tracheal rigidity was found at the repair site. Outer diameter measurements of tracheal rings 1, 4, and 8 (corresponding to levels above, at, and below the repair site) revealed substantial growth of the repaired segment with a slight narrowing when compared to surrounding normal trachea (see Table 2). Inner diameter measurements at these levels revealed a narrowing within the repaired region ranging from 2 to 4 mm.

Histopathologic examination revealed that the tracheal wall in the area of the implant was more rigid than normal fixed trachea. Tracheal ulceration, fissures, or protrusion of prosthetic material into the lumen was not seen. Microscopic examination showed the empty spaces, previously occupied by the trabeculae of the PLPG stent, external to the tracheal cartilage segments. These angulated spaces were lined by myofibroblastic and collagenized fibrous tissue with a scant lymphocytic infiltrate. The thickness of this fibrous layer varied from 0.25 to 2.0 mm. The

TABLE 2
OUTER TRACHEAL DIAMETERS BEFORE
OPERATION AND AT END OF OBSERVATION PERIOD

| | | Preoperative | Final |
|---|---|---|---|
| Pig | Tracheal Ring | Diameter (mm) | Diameter (mm) |
| 1 | 1 | 14 | 21 |
| | 4 | 14 | 20 |
| | 8 | 14 | 22 |
| 2 | 1 | 14 | 22 |
| | 4 | 15 | 20 |
| | 8 | 14 | 22 |
| 3 | 1 | 14 | 20 |
| | 4 | 14 | 18 |
| | 8 | 14 | 19 |
| 4 | 1 | 15 | 21 |
| | 4 | 16 | 20 |
| | 8 | 16 | 22 |
| 5 | 1 | 14 | 21 |
| | 4 | 14 | 20 |
| | 8 | 14 | 21 |
| 6 | 1 | 14 | 20 |
| | 4 | 15 | 19 |
| | 8 | 15 | 20 |

submucosa contained the normal areas of adipose tissue, smooth muscle components, and seromucinous glands. In many sections, a normal epithelium with abundant cilia was present. In some areas, the mucosal epithelium was flat and attenuated to three cell layers in thickness; however, some cilia were still evident in most of these sampled areas. Squamous metaplasia was not seen.

During creation of the tracheomalacia model, there were two animal deaths due to severe postoperative wound infections. These deaths occurred on postoperative days 6 and 7, respectively. In both cases, the animals were witnessed having contaminated their incision lines after removing their surgical dressings or having their dressings removed by other animals. There was no evidence of respiratory distress in either animal prior to death, and examination of the airway during necropsy revealed a secure stent without evidence of airway collapse. As a result of these deaths, additional measures were taken to protect the surgical wound, including the use of multiple layers of dressings, postoperative broad spectrum antibiotics, and housing of the animal in an isolated environment for one week after surgery. Following institution of these measures, no further wound infections or deaths were encountered.

What is claimed is:

1. An exoluminal stent comprising: a bioabsorbable matrix having a plurality of apertures passing therethrough, wherein the matrix is capable of being dimensioned and configured to complement an external diameter of a vessel to be stented.

2. The exoluminal stent of claim 1, wherein the bioabsorbable matrix comprises poly(lactic acid).

3. The exoluminal stent of claim 1, wherein the bioabsorbable matrix comprises poly(glycolic acid).

4. The exoluminal stent of claim 1, wherein the bioabsorbable matrix comprises a poly(lactic acid)/poly(glycolic acid) copolymer.

5. The exoluminal stent of claim 4, wherein the poly(lactic acid)/poly(glycolic acid) co-polymer comprises from about 50 to about 90 wt % lactic acid monomers and from about 50% to about 10% glycolic acid monomers.

6. The exoluminal stent of claim 4, wherein the poly(lactic acid)/poly(glycolic acid) co-polymer comprises from about 80 to about 90 wt % lactic acid monomers and from about 20% to about 10% glycolic acid monomers

7. The exoluminal stent of claim 1, wherein the bioabsorbable matrix comprises a desaminotyrosyltyrosine ethyl ester.

8. The exoluminal stent of claim 1, wherein the bioabsorbable matrix comprises a homopolymer selected from the group consisting of poly(lactic acid), poly(glycolic acid), poly(caprolactone) and polydioxanone.

9. The exoluminal stent of claim 1, wherein the bioabsorbable matrix comprises a copolymer selected from the group consisting of poly(lactic acid)/(glycolic acid) copolymer, poly(ε-caprolactone)/(lactide) copolymer, and poly(ε-caprolactone)/(glycolide) copolymer.

10. An exoluminal stent comprising: an absorbable matrix comprising a poly(lactic acid)/poly(glycolic acid) co-polymer, the matrix having a plurality of apertures passing therethrough, and wherein the matrix is capable of being dimensioned and configured to complement an external diameter of a vessel to be stented.

11. The exoluminal stent of claim 10, wherein the poly(lactic acid)/poly (glycolic acid) co-polymer comprises from about 50 to about 90 wt % lactic acid monomers and from about 50% to about 10% glycolic acid monomers.

12. The exoluminal stent of claim 10, wherein the poly(lactic acid)/poly(glycolic acid) co-polymer comprises from about 80 to about 90 wt % lactic acid monomers and from about 20% to about 10% glycolic acid monomers

13. A method of externally stenting a vessel in need thereof, the method comprising:
to an external diameter of a vessel to be stented, adhering a bioabsorbable matrix having a plurality of apertures passing therethrough, wherein the matrix is dimensioned and configured to complement the external diameter of the vessel.

14. The method of claim 13, wherein the bioabsorbable matrix comprises poly(lactic acid).

15. The method of claim 13, wherein the bioabsorbable matrix comprises poly(glycolic acid).

16. The method of claim 13, wherein the bioabsorbable matrix comprises a poly(lactic acid)/poly(glycolic acid) copolymer.

17. The method of claim 16, wherein the poly(lactic acid)/poly(glycolic acid) co-polymer comprises from about 50 to about 90 wt % lactic acid monomers and from about 50% to about 10% glycolic acid monomers.

18. The method of claim 16, wherein the poly(lactic acid)/poly(glycolic acid) co-polymer comprises from about 80 to about 90 wt % lactic acid monomers and from about 20% to about 10% glycolic acid monomers

19. The method of claim 13, wherein the bioabsorbable matrix comprises a desaminotyrosyltyrosine ethyl ester.

20. The method of claim 13, wherein the bioabsorbable matrix comprises a homopolymer selected from the group consisting of poly(lactic acid), poly(glycolic acid), poly(caprolactone) and polydioxanone.

21. The method of claim 13, wherein the bioabsorbable matrix comprises a copolymer selected from the group consisting of poly(lactic acid)/(glycolic acid) copolymer, poly(ε-caprolactone)/(lactide) copolymer, and poly(ε-caprolactone)/(glycolide) copolymer.

22. The method of claim 13, wherein the vessel to be stented is a trachea.

23. The method of claim 22, wherein the bioabsorbable matrix is adhered to the trachea via sutures passing through the apertures in the matrix.

24. The method of claim 13, wherein the bioabsorbable matrix is adhered to the vessel via sutures passing through the apertures in the matrix.
